(19) United States
(12) Patent Application Publication
(10) Pub. No.: US 2026/0216486 A1
(43) Pub. Date: Jul. 30, 2026

(54) ENTEROCUTANEOUS FISTULA MANAGEMENT DEVICE

(71) Applicants: CASE WESTERN RESERVE UNIVERSITY, Cleveland, OH (US); UNIVERSITY HOSPITALS CLEVELAND MEDICAL CENTER, Cleveland, OH (US)

(72) Inventors: Jeffrey Marks, Cleveland, OH (US); Steve Schomisch, Cleveland, OH (US)

(21) Appl. No.: 19/147,030

(22) PCT Filed: Jan. 10, 2024
(86) PCT No.: PCT/US2024/011056

§ 371 Date: Jul. 10, 2025

Related U.S. Application Data
(60) Provisional application No. 63/479,303, filed on Jan. 10, 2023.

(51) Int. Cl. A61M 27/00; A61B 17/00; A61B 17/12; A61M 25/00; A61M 25/10

(52) U.S. Cl. CPC A61M 27/002; A61M 25/005; A61M 25/1011; A61B 2017/00641; A61B 17/12045; A61M 2205/3331; A61M 2210/1042

(57) ABSTRACT

A device for bypassing an enterocutaneous fistula in a patient includes a flexible tube extending from a first axial end to a second axial end and defining a passage extending the entire length of the tube, inflatable cuffs located at each of the first axial end and the second axial end of the tube, the inflatable cuffs configured to seal the first axial end and the second axial end to the internal wall of a bowel, at least one inflation tube fluidly connected to the inflatable cuffs for supplying inflation liquid to and from the inflatable cuffs, the inflatable cuffs are configured to inflate to a fluid pressure effective to create a fluid seal to the internal wall of the bowel.

### RELATED APPLICATION

This application claims priority from U.S. Provisional Application No. 63/749,303, filed Jan. 10, 2023, the subject matter of which is incorporated herein by reference in its entirety.

### TECHNICAL FIELD

The present invention relates generally to medical devices, and specifically to an enterocutaneous fistula management bypass device for treating an enterocutaneous fistula.

### BACKGROUND

An enterocutaneous fistula (ECF) is a pathological condition where a connection forms between the intestinal tract and the skin. As a result, intestinal contents leak onto the skin. Most ECFs occur after bowel surgery but can also occur due to infection, inflammatory bowel disease, Crohn's disease, or traumatic injury to the bowel. Leakage of enteral contents results in dehydration, malnutrition, risk of sepsis, and wound complications. Because of a poorly understood pathological etiology, management of patients with ECF is difficult requiring a multidisciplinary approach. Surgical reconstruction is frequently attempted, but it is widely recognized that this should be performed later rather than sooner. Early post-operative management strategies generally involve control of infection and sepsis, fluid replacement, nutritional supplementation and control of enteral leakage often using standard ostomy devices, and wound and skin care.

Nonsurgical management of the ECF output is limited to the use of devices designed for surgically created ostomies. These devices are intended to protect the skin and collect the leakage. This approach does not diminish the output nor the need for fluid and nutritional replacement. A better approach would be to restore the bowel continuity across the ECF-thereby decreasing output (and wound-related complications) and reducing the need for fluid and nutritional replacement. While the off-label use of self-expanding metal stents to bypass the area of fistulization have been attempted, the success rate of transabdominal stenting is very low given the limitations of these stents. Common limitation of transabdominal stenting may include the inability to adequately seal to the bowel wall, the use of excessive radial force for stabilization, the inability to conform to the dynamic segment of the GI tract, migration away from the fistula site and permanence (inability to remove the stent). Moreover, the metallic and rigidly straight characteristics of these stents frequently result in mucosal erosions or occlusion. Prior attempts to design an ECF bypass device may have failed due to excessive radial forces. Inflatable balloons or cuffs used to seal to the bowel must not disrupt normal blood perfusion of the tissue. For example, a standard Foley balloon catheter (30 cc) when inflated to just 10 ml can reach radial pressures in excess of 250 mmHg which will cause decreased blood perfusion leading to ischemia and disruptions to peristalsis and mucosal secretion. Therefore, there remains a need for devices specifically intended to address the complications of ECF, ideally retaining the enteral contents, protecting the skin and thereby, potentially limiting the need for nutritional supplementation.

### SUMMARY

This disclosure relates to an enterocutaneous fistula management device for placement in the bowel spanning the fistula opening to allow enteral contents to bypass the fistula tract. The device includes a flexible tube that extends from a first axial end to a second axial end and that defines a passage extending the entire length of the tube. The device also includes inflatable cuffs located at each of the first axial end and the second axial end of the tube. The inflatable cuffs are configured to seal the first axial end and the second axial end to an internal wall of a bowel. The inflatable cuffs are configured to produce adequate surface area with lower radial forces enabling a seal to the bowel wall while allowing for normal blood perfusion. The device also includes at least one inflation tube fluidly connected to the inflatable cuffs for supplying inflation liquid to and from the inflatable cuffs. The inflatable cuffs are configured to inflate to a fluid pressure effective to create a fluid seal to the internal wall of the bowel. The device further includes an auxiliary tube fluidly connected with the flexible tube. The auxiliary tube extends from the flexible tube at a location between the first axial end and the second axial end. The device also includes a support wire that engages an inner surface of the flexible tube and extends axially along the inner surface between the first axial end and the second axial end.

In some embodiments, the flexible tube can have a length of about 10.0 cm to about 20.0 cm, an outer diameter of about 8.0 mm to about 26.0 mm, and/or an inner lumen diameter of about 6.0 mm to about 20.0 mm. In some embodiments, the flexible tube is a flexible silicone tube. The flexible tube can have a solid or fluid impermeable wall preventing radial flow of fluid through the wall of the tube.

In some embodiments, the flexible tube first axial and/or second axial end are shaped with blunt or square edge(s).

In some embodiments, the flexible tube first axial end and/or second axial end are shaped with beveled tip(s) to increase the area of the opening reducing risk of bowel obstruction.

In some embodiments, the flexible tube first axial end and/or second axial end are shaped in a distinct pattern, such as a wavy edge or end forming two, three, or more protrusions or lobes creating leading edges to reduce risk of bowel obstruction.

In some embodiments, the inflatable cuffs provide a fluid barrier against the internal bowel wall while substantially minimizing ulceration or abrasion of the bowel. The inflatable cuffs can be configured to provide an internal fluid pressure of about 30 mmHg to about 50 mmHg at a volume of about 20 mL when the device is implanted and confined in the bowel. In some embodiments, the inflatable cuffs can expand without a substantial increase in internal fluid pressure.

In some embodiments, the fluid pressure effective to create a fluid seal to the internal wall of the bowel allows for substantially normal intestinal blood perfusion, peristalsis, and mucosal secretion in the bowel.

In some embodiments, the inflatable cuffs can be configured to distribute a substantially equal radial force to the internal wall of the bowel in contact with the inflatable cuffs.

In some embodiments, the inflatable cuffs can extend axially about 20 mm to about 60 mm along the outer surface of the first axial end and the second axial end. The inflatable cuffs can be configured to seal the first axial end to the internal wall of the bowel upstream of the fistula and to seal the second axial end to the internal wall of the bowel downstream of the fistula.

In some embodiments, the at least one inflation tube extends from the inflatable cuffs along the passage of the flexible tube and radially from the outer surface of the flexible tube at about the midpoint between the first axial end and the second axial end.

The at least one inflation tube can have a port fluidly connected to the inflation cuffs. The port can include a valve and a connector. Optionally, the at least one inflation tube can include a manometer.

In some embodiments, the device can include a first inflation tube fluidly connected to a first inflatable cuff and a second inflation tube fluidly connected to a second inflatable cuff.

In some embodiments, the auxiliary tube has an external port fluidly connected to the flexible tube passage. The external port can include a connector. In some embodiments, the auxiliary tube extends from the flexible tube at about the midpoint between the first axial end and the second axial end, preferably, the auxiliary tube extends from the flexible tube at a location between the inflation cuffs.

In some embodiments, the at least one inflation tube and the auxiliary tube are positioned substantially parallel to a centerline and against an outer surface of the flexible tube. Positioning the at least one inflation tube and the auxiliary tube substantially parallel to a centerline and against an outer surface of the flexible tube can facilitate insertion of the device in a unidirectional fashion in the bowel without folding of the device.

In some embodiments, the support wire is a wire coil provided within the passage of the flexible tube. The support wire can include medical grade stainless steel. The support wire coil can have a pitch of about 0.5 mm to about 10 mm.

In some embodiments, the support wire can be made from shape memory material or alloy, such as nitinol.

In some embodiments, the support wire can be configured in a pattern to allow the support wire to move from an expanded position to a collapsed or insertion position. The collapsed or insertion position can decrease the outer diameter of the flexible tube. Elongation, compression, and/or collapse of the support wire can decrease the outer diameter of the flexible tube prior to insertion of the device through the fistula. The support wire can also be configured in an expandable pattern to increase the outer diameter of the flexible tube after insertion into the bowel.

In some embodiments, the flexible tube, the at least one inflation tube, the auxiliary tube and/or the inflatable cuffs are formed from a material having sufficient elasticity to allow the flexible tube, the at least one inflation tube, the auxiliary tube and/or the inflatable cuffs to stretch in a manner substantially similar to the elongation of the collapsed support wire and expansion of the expanded support wire.

In some embodiments, the entire device can be elongated, compressed, and/or collapsed to allow insertion through an opening and then expanded to original device dimensions. The device being elongated, compressed, and/or collapsed allows for the device to be inserted through an opening smaller than a device having the original dimensions.

Other embodiments relate to a method of managing fistula output using the fistula management device described herein. The method includes the steps of: (a) inserting the first and second axial ends of the flexible tube in a bowel via a fistula opening such that: (i) a first section of the bowel located upstream of the fistula opening overlaps a first inflatable cuff; and (ii) a second section of the bowel located downstream of the fistula opening overlaps a second inflatable cuff; and (b) creating a fluid seal between each of the first inflatable cuff, second inflatable cuff, and the bowel by inflating the first and second inflatable cuffs to sizes, e.g., circumferences or diameters, effective to engage the internal wall of the bowel.

In some embodiments, the inflatable cuffs are inflated to a fluid pressure effective to create a fluid seal while preventing ischemia. In some embodiments, the inflatable cuffs can be inflated using fluoroscopic or manometry guidance where the fluid pressure of the inflatable cuffs can be monitored during inflation.

In some embodiments, the device can be inserted in the bowel of a patient manually, for example, using a wire-guided dilation balloon.

In some embodiments, prior to the step (a), the method further includes the step of positioning the at least one inflation tube and the auxiliary tube substantially parallel to a centerline and against an outer surface of the flexible tube to facilitate insertion of the device in a unidirectional fashion in the bowel without folding of the device.

In other embodiments, following step (a), the method further includes repositioning the device within the bowel to align the midpoint of the flexible tube with the fistula opening using the at least one inflation tube and the auxiliary tube.

In some embodiments, following step (b), the method further includes providing an external bumper configured to retain the at least one inflation tube and the auxiliary tube through the fistula opening at a patient's skin and prevent migration of the device.

In other embodiments, following step (b), the method further includes removing the device by deflating the inflatable cuffs and withdrawing the device through the fistula opening.

In other embodiments, following step (b), the method further includes removing the device by deflating the inflatable cuffs, severing the at least one inflation tube, and auxiliary tube and allowing the device to pass through the bowel.

In some embodiments, the support wire can be removed in a manner which softens the flexible tube to allow the device to pass through the bowel or be removed through the fistula opening.

Other objects and advantages and a fuller understanding of the invention will be had from the following detailed description and the accompanying drawings.

### BRIEF DESCRIPTION OF THE DRAWINGS

FIG. 1 is a schematic illustration of an example enterocutaneous fistula (ECF) management bypass device.

FIG. 2 is a section view of the bypass device of FIG. 1 taken along line 22.

FIG. 3 is a schematic illustration of an example ECF management bypass device implanted in the gastrointestinal tract.

FIG. 4 is a schematic illustration of an enterocutaneous fistula.

FIG. 5 is a photographic image of an example ECF management bypass device showing inflation and auxiliary tubes fitted with valves and luer lock connectors and extending from the primary tube of the device.

FIG. 6 is a photographic image of a side view of the example ECF management bypass device showing supporting wire coil within the primary tube of the device.

FIG. 7 is a photographic image of an example ECF management bypass device showing the primary tube lumen, and the inflation and auxiliary tubes with valves and luer lock connectors.

FIG. 8 is a photographic image of a portion of an example ECF management device with one inflated cuff confined within a tube and having an elongated shape with a rounded end.

FIG. 9 is another photographic image of the portion of an example ECF management device with one inflated cuff confined within a tube and having an elongated shape with a rounded end.

FIG. 10 is a photographic image of an example ECF management device preloaded with a wire-guided balloon for insertion.

FIG. 11 is a photographic image showing an example ECF management device preloaded with an inflated wire-guided balloon beginning to be inserted into a tube with a hole-simulating fistula insertion.

FIG. 12 is a photographic image showing an example ECF management device preloaded with an inflated wire-guided balloon inserted midway into a tube with a hole-simulating fistula insertion.

FIG. 13 is a photographic image showing an example ECF management device preloaded with an inflated wire-guided balloon entirely inserted into a tube with hole-simulating fistula insertion.

FIG. 14 is a photographic image showing an example ECF management device entirely inserted into a tube with hole-simulating fistula insertion where the wire-guided balloon is removed.

FIG. 15 is a photographic image showing an example ECF management device entirely inserted into a tube with hole-simulating fistula insertion and repositioned to center the device over the hole

FIG. 16 is a schematic drawing of an axial end of an ECF device having wavy edge.

### DETAILED DESCRIPTION

To facilitate the understanding of this invention, a number of terms are defined below. Terms defined herein have meanings as commonly understood by a person of ordinary skill in the areas relevant to the present invention.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of ±20%, ±15%, ±10%, ±9%, ±8%, ±7%, ±6%, ±5%, ±4%, ±3%, ±2%, or ±1%.

Embodiments descried herein relate to medical devices for repairing a fistula, and specifically to an enterocutaneous fistula (ECF) management bypass device for treating an enterocutaneous fistula.

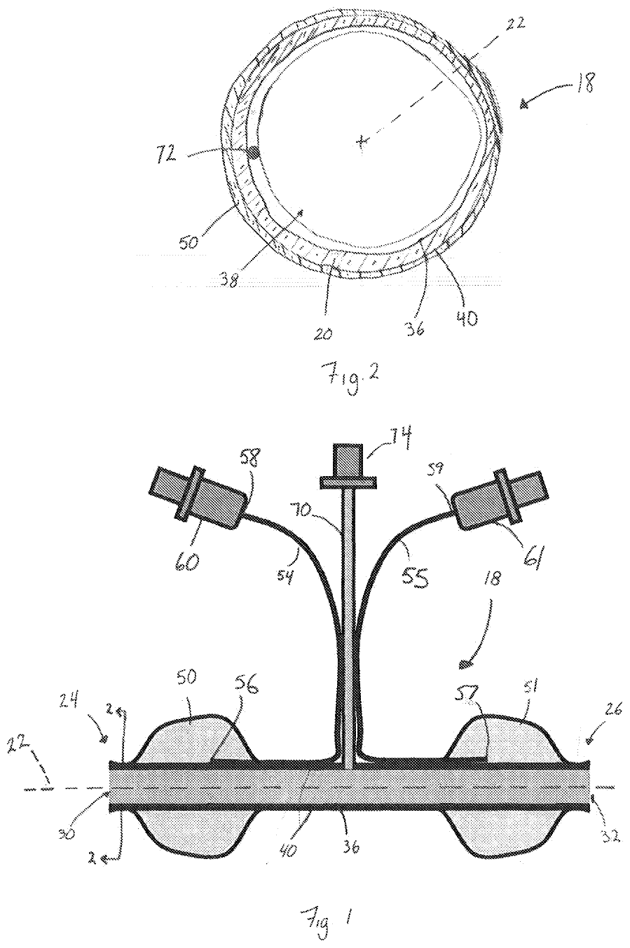

FIGS. 1-4 illustrate an ECF management bypass device 18 in accordance with an aspect of the invention. The ECF management bypass device 18 (or simply, bypass device) includes a flexible tube 20 extending along an axis or centerline 22 from a first axial end 24 to a second axial end 26. The flexible tube 20 of the ECF management bypass device 18 is designed to be inserted into a portion of a GI tract 82, i.e., into a bowel 84 lumen, via a fistula tract opening 90 such that a first section of the bowel 85 located upstream of the fistula opening 90 overlaps the first axial end 24 and a second section of the bowel 87 located downstream of the fistula opening 90 overlaps the second axial end 26.

The tube 20 can have a round, e.g., circular, longitudinal cross-section, that extends from a first opening 30 provided at the first axial end 24 to a second opening 32 provided at the second axial end 26. The tube 20 includes an outer surface 40 and an inner surface 36 that defines a passage or lumen 38 extending the entire length of the tube 20 and fluidly connecting the openings 30, 32 to one another. The tube 20 wall can be solid or fluid impermeable such that fluid only flows through the passage 38 and generally along the centerline 22, i.e., not radially out of the tube 20 or tube wall.

The tube 20 can be formed from a biocompatible, flexible material, such as a polymer. The material is selected to ensure the tube 20 is waterproof, durable, conformable, and capable of withstanding and contacting bile, enzymes, chyme, etc. in the GI tract 82. To this end, the tube 20 can include folds or kinks (not shown) to facilitate bending of the tube through the GI tract 82.

Examples of polymers that can be used to form the tube 20 include, but are not limited to, silicone, fluoropolymer, rubber, or cellophane. The polymer can preferably have a tensile strength capable of resisting tearing or destruction of the tube 20 during implantation and deformation of the device 18. In certain embodiments, the polymer can be an elastic silicone, e.g., a platinum cured silicone. The elastic silicone polymer forming the tube 20 can have a shore hardness ranging from about 10 A to about 50 A, about 20 A to about 40 A, or about 25 A to about 35 A. In an exemplary embodiment, the tube 20 is formed from a platinum cured silicone material having a shore hardness of 30 A, a tensile strength of 500 psi, an elongation at break of 364%, and a tear strength (Die B) of 108 pli. Optionally, the tube 20 can include radiopaque markers to enable visualization of anatomical location of the tube 20 with fluoroscopy.

Advantageously, the flexible tube 20 is thin walled such that the outer diameter of the tube 20 is minimally larger than the inner diameter. The radial thickness of the tube 20 wall between the inner and outer surfaces 36, 40 should be selected to enable the tube 20 to collapse under its own weight, i.e., when there is no fluid pressure within the passage 38 and expand in response to very low fluid pressure. In some embodiments, the tube 20 can have a wall thickness of about 0.2 mm to about 1.0 mm, for example, about 0.3 mm to about 1.0 mm, about 0.4 mm to about 1.0 mm, about 0.5 mm to about 1.0 mm, about 0.6 mm to about 1.0 mm, about 0.7 mm to about 1.0 mm, about 0.8 mm to about 1.0 mm, about 0.9 mm to about 1.0 mm, about 0.2 mm to about 0.9 mm, about 0.2 mm to about 0.8 mm, about 0.2 mm to about 0.7 mm, about 0.2 mm to about 0.6 mm, about 0.2 mm to about 0.5 mm, about 0.2 mm to about 0.4 mm, about 0.2 mm to about 0.3 mm, about 0.3 mm to about 0.9 mm, or about 0.4 mm to about 0.6 mm. In one example, the tube 20 can have a wall thickness of about 0.5 mm.

In some embodiments, the tube 20 can have an outer diameter of about 8.0 mm to about 26.0 mm, for example, about 10.0 mm to about 26.0 mm, about 12.0 mm to about 26.0 mm, about 14.0 mm to about 26.0 mm, about 16.0 mm to about 26.0 mm, about 18.0 mm to about 26.0 mm, about 20.0 mm to about 26.0 mm, about 22.0 mm to about 26.0 mm, about 24.0 mm to about 26.0 mm, about 8.0 mm to about 24.0 mm, about 8.0 mm to about 22.0 mm, about 8.0 mm to about 20.0 mm about 8.0 mm to about 18.0 mm, about 8.0 mm to about 16.0 mm, about 8.0 mm to about 14.0 mm, or about 8.0 mm to about 16.0 mm. In one example, the tube 20 can have an outer diameter of about 12.0 mm.

In some embodiments, the tube 20 can have an inner diameter of about 6.0 mm to about 20.0 mm. In one example, the tube 20 can have an inner diameter of about 10.0 mm.

The tube 20 can have a length effective to allow the device 18 when confined in the bowel 84 to extend from a position in the bowel 84 upstream of a fistula 90 to a position of the bowel 84 downstream of the fistula. The length of the tube 20 can vary depending on the desired use or placement of the device 18 in a patient 80. In some embodiments, the tube 20 can have a length ranging from about 10.0 cm to about 20.0 cm, for example, about 10.0 cm to about 18.0 cm, about 10.0 cm to about 16.0 cm, about 10.0 cm to about 14.0 cm, about 10.0 cm to about 12.0 cm, about 12.0 cm to about 20.0 cm, about 14.0 cm to about 20.0 cm, about 16.0 cm to about 20.0 cm, or about 18.0 cm to about 20.0 cm. In one example, the tube 20 can have a length of about 10 cm.

The tube 20 may be configured to resist collapsing inward when subjected to external forces of up to about 250 mmHg when implanted in the bowel 84 of a patient 80. The tube 20 can have an inner tube burst pressure less than about 250 mmHg as the pressure of the intestinal tract when the device 18 is implanted and confined in the bowel 84 is typically significantly less than 250 mmHg.

In one embodiment of the device 18, the first axial end 24 and/or the second axial end 26 of the tube can have an eyelet to which a wire, thread, or suture can be affixed for safe guidance of the device during placement or removal of the device 18 through the fistula opening 90.

In some embodiments, the flexible tube first axial end 24 and/or second axial end 26 are shaped with blunt or square edge(s) (not shown).

In some embodiments, the flexible tube first axial end 24 and/or second axial end 26 are shaped with beveled tip(s) (not shown) to increase the area of the opening reducing risk of bowel obstruction.

In some embodiments, the flexible tube first axial end 24 and/or second axial end are shaped in a distinct pattern, such as a wavy edge or end forming two, three, or more protrusions or lobes creating leading edges to reduce risk of bowel obstruction. Referring to FIG. 16, which is a schematic view of the first axial end 24, the first axial end 24 can have a wavy axially extending, circumferential edge 100. By “wavy” it is meant a series of undulating and wavelike curves. The wavy edge 100 may undulate in a sinusoidal shape and may include at least two, three, or more axially extending excurvate lobes between axially extending incurvate portions 104 or valleys. The wavy edge 100 need not include sharp or jagged portions, for instance, intersecting straight angled edges, straight angled edges intersecting rounded edges, or the like, which may facilitate ripping or tearing of the first axial end 24 of flexible tube 20 thereby reducing risk of bowel obstruction. Rather the wavy edge 100 may be smoothly continuously curving to create leading edges to reduce risk of bowel obstruction.

Referring again to FIGS. 1-3, the bypass device 18 further includes an elongated first inflatable cuff 50 and an elongated second inflatable cuff 51 located, respectively, at the first axial end 24 and the second axial end 26 of the tube 20. The first and second inflatable cuffs 50, 51 surround or are positioned circumferentially around the first axial end 24 and the second axial end 26 and are configured to seal the first axial end 24 and the second axial end 26 to the internal wall 89 of a bowel 84. Inflation of the inflatable cuffs 50, 51 creates a fluid seal between first and second cuffs 50, 51 and the internal wall 89 of the bowel 84. After the fluid seal is created enteric contents 92 flowing through the bowel 84 are directed through the main fluidic passage 38 of the flexible tube 20 and beyond the location of the fistula opening 90 in the bowel 84. Accordingly, use of the fistula management device 18 can effectively prevent enteric contents 92 from leaking out of a bowel 84 through a fistula 90 opening in the bowel 84.

In an exemplary embodiment, the elongated first and second inflatable cuffs 50, 51 are configured to seal the first axial end 24 to the internal wall 89 of the bowel 84 upstream of a fistula 90 and the second axial end 26 to the internal wall 89 of the bowel 84 downstream of the fistula 90. The first and second inflatable cuffs 50, 51 can provide a fluid barrier against the internal bowel wall 89 while substantially minimizing ulceration or abrasion of the bowel 84.

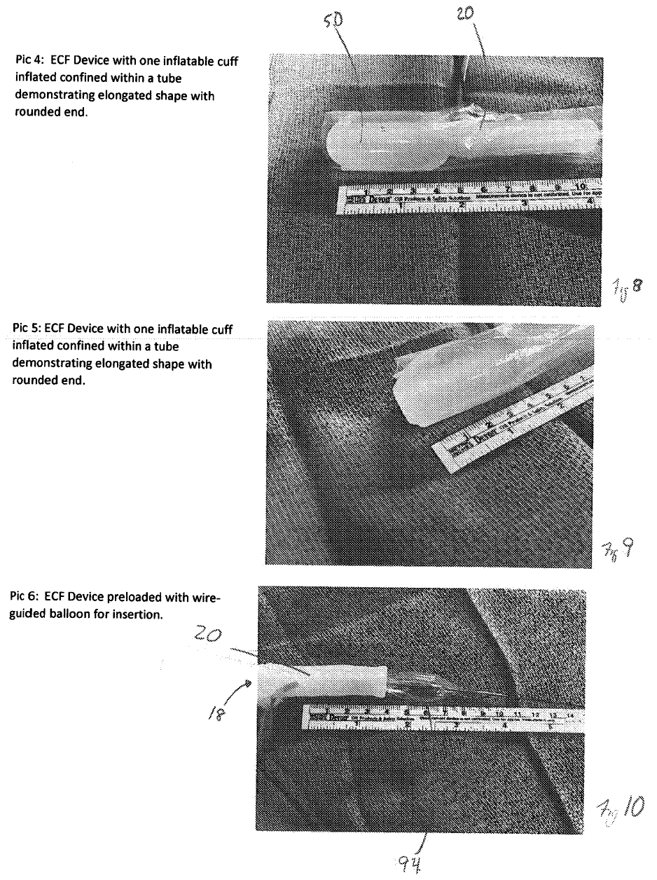

The first and second inflatable cuffs 50,51 are elongated and have a substantially smooth outer surface in order to provide a sufficiently large surface area to contact the internal wall 89 of the bowel 84 and create a seal. As shown in FIGS. 8-9, the first and second inflatable cuffs 50, 51 when inflated and contained in the bowel 84 can also have a smooth rounded tapered edge to reduce risk of bowel injury.

In some embodiments, the first and second inflatable cuffs 50, 51 can extend axially about 20 mm to about 60 mm, for example, about 25 mm to about 60 mm, about 30 mm to about 60 mm, about 35 mm to about 60 mm, about 40 mm to about 60 mm, about 45 mm to about 60 mm, about 20 mm to about 55 mm, about 20 mm to about 50 mm, or about 20 mm to about 40 mm along the outer surface 40 of the first axial end 24 and the second axial end 26. In some embodiments, each of the inflatable cuffs 50, 51 can have an axial length of about 45 mm to about 55 mm. In some embodiments, each of the inflatable cuffs 50, 51 are about 50 mm in length.

The first and second inflatable cuffs 50, 51 can be fabricated from highly elastic materials and when inflated can form a fluid seal between the tube 20 and the walls 89 of a bowel lumen. The first and second inflatable cuffs 50, 51 can also expand without a substantial increase in internal fluid pressure. For example, an initial expansion of inflatable cuffs from about 3 ml to about 4 ml of volume may create a step in pressure of about 15-20 mmHg and an expansion from 4 ml through 20 ml of volume may create a pressure rise of only about an additional 2-4 mmHg. Similar to the flexible tube 20, the inflatable cuffs 50, 51 can be configured to have a burst pressure (or burst strength) of less than about 250 mmHg.

In some embodiments, the inflatable cuffs 50, 51 can be co-cast along with the primary tube 20 to reduce overall outer diameter of the device 18. Co-casting the inflatable cuffs 50, 51 with the tube 20 can allow the cuffs 50, 51 to be folded back along the primary tube 20 to create the softer, rounded edges for the first axial end 24 and the second axial end 26 of the device 18 that can potentially reduce the risk of ulceration or abrasion of the bowel 84.

In some embodiments, the wavy ends are affixed to the first axial end 24 and the second axial end 26.

In some embodiments, the wavy ends are co-cast with the casting of the primary tube 20.

In some embodiments, the inflatable cuffs 50, 51 can be formed from a polymer, including but not limited to silicone, fluoropolymer, rubber, or cellophane. In some embodiments, the polymer is an elastic silicone, e.g., a platinum cure silicone. In certain embodiments, the first and second inflatable cuffs 50, 51 can be formed from a thin low durometer silicone material. The first and second inflatable cuffs 50, 51 can have a shore hardness ranging from 00-10 A to about 00-50 A. In an exemplary embodiment, the first and second inflatable cuffs 50, 51 are formed from a platinum cure silicone material having a shore hardness of 00-10 A, a tensile strength of 120 psi, an elongation at break of 800%, and a tear strength (Die B) of 22 pli.

The first and second inflatable cuffs 50, 51 are preferably configured to distribute a substantially low and equal radial force along and against the internal wall of the bowel 89 in contact with the inflatable cuffs 50,51. Low radial forces generated by the first and second inflatable cuffs 50, 51 are less likely to cause ischemia leading to ulceration and more likely to allow for normal intestinal functions such as blood perfusion, peristalsis, and mucosal secretions. Therefore, the inflatable cuffs 50,51 can be configured to be inflated when confined in the bowel 84 to an internal fluid pressure that provides an adequate seal while not creating ischemia of the bowel tissue and/or allows for normal intestinal functions such as blood perfusion, peristalsis, and mucosal secretions.

In some embodiments, the first and second inflatable cuffs 50, 51 when inflated are configured to allow the bowel 84 to deform the inflatable cuffs 50, 51 rather than the cuffs causing deformation of the bowel 84. The first and second inflatable cuffs 50, 51 when inflated and constrained by the bowel wall 89 can also primarily elongate rather than expand radially. The longitudinal expansion can increase the surface area of the inflatable cuffs 50, 51 in contact with the bowel 84, thereby allowing the inflatable cuffs 50, 51 to provide an improved fluid barrier with lower radial force on the bowel wall 89.

The first and second inflatable cuffs 50, 51 can be inflated to a pressure effective to form a seal to the bowel wall 89. The inflatable cuffs' 50, 51 diameters can substantially match the bowel 84 diameter while applying an amount of radial force to the bowel 84 effective to form a seal with the bowel wall 89. For example, the diameter of each of the inflatable cuffs 50, 51 when inflated can be substantially equal to the internal diameter of the bowel 84 while applying up to about 50 mmHg of radial force on the internal wall of the bowel 89.

The pressure to which the cuffs 50, 51 are inflated may vary depending on inflated volume of the cuffs 50, 51. In some embodiments, the first and second inflatable cuffs 50, 51 can be inflated to provide an internal fluid pressure of about 15 mmHg to about 50 mmHg at a volume of about 20 ml. For example, the inflatable cuffs 50, 51 can be inflated to provide an internal fluid pressure of about 35 mmHg to about 45 mmHg at a volume of about 20 ml, about 40 mmHg at a volume of about 20 ml, about 15 mmHg to about 35 mmHg at a volume of about 10 ml, or about 15 mmHg to about 30 mmHg at a volume of about 10 ml.

When deflated, the diameters of the first and second inflatable cuffs 50, 51 do not substantially contribute to the radial diameter of the flexible tube 20. In an exemplary embodiment, the diameter of the inflatable cuffs 50, 51 add about 0.5 mm to the total outer diameter of the tube 20. However, upon inflation, the diameter of each of the inflatable cuffs 50, 51 is dependent on the confinement of the individual cuff. Unconfined, the diameter of the cuffs 50, 51 is larger than when confined in the bowel 84.

It will be appreciated that one or more inflatable cuffs in addition to the first and second inflatable cuffs 50, 51 can be provided along the length of the tube 20, e.g., at the first axial end 24 and/or the second axial end 26, to further help seal the tube to the bowel wall 89.

The bypass device 18 also includes a first inflation tube 54 and a second inflation tube 55 fluidly connected to the first and second inflatable cuffs 50, 51 to supply inflation fluid, such as a liquid or gas, to and release inflation fluid from the first and second inflatable cuffs 50, 51. The first and second inflation tubes 54, 55 can include silicone tubing that is inserted into the first and second inflatable cuffs 50, 51 and secured with a silicone adhesive. The first and second inflation tubes 54, 55 can extend from the first inflatable cuff 50 and second inflatable cuff 51 along the passage 38 of the flexible tube 20 and radially from the outer surface 40 of the flexible tube 20 at about a midpoint between the first axial end 24 and the second axial end 26. It will be appreciated that while two separate inflation tubes 54, 55 are illustrated, the device may employ a single inflation tube that is fluidly connected to the inflatable cuffs 50, 51.

Referring again to FIG. 1, the first inflation tube 54 can include a first end 56 fluidly connected to the first cuff 50 and a second end 58 having a port 60 for receiving/releasing the inflation fluid to selectively inflate/deflate the first cuff 50. The inflation tube 54 can be capped with a valve to retain fluid pressure and/or a connector, such as a luer connector. Similarly, the second inflation tube 55 can include a first end 57 fluidly connected to the cuff 51 and a second end 59 having a port 61 for receiving/releasing the inflation fluid to selectively inflate/deflate the second cuff 51. In additional embodiments, a syringe may be inserted into the ports 60, 61 to supply an inflation fluid, such as a liquid or gas, to the first and second inflatable cuffs 50, 51 via the first and second inflation tubes 54, 55.

The first and second inflation tubes 54, 55 can be formed from a biocompatible plastic intended for medical use, such as but not limited to medical grades of silicone, PVC, polyethylene, polyethersulfone (PES), polycarbonate, polyetheretherketone (PEEK), polytetrafluoroethylene (PTFE), polyetherimide (PEI), polypropylene, polysulfone and polyurethane. The first and second inflation tubes 54, 55 can have an outer diameter of about 1.0 mm to about 3.0 mm. In certain embodiments, the first and second inflation tubes 54, 55 can have an outer diameter of about 2.0 mm.

When confined in the bowel 84 the first and second inflatable cuffs 50, 51 cannot be readily visualized without the use of repeated fluoroscopic imaging to assess cuff inflation status. Therefore, in some embodiments, the first and second inflation tubes 54, 55 can further include a manometer (not shown), such as a manometry balloon, to indicate the inflation status of the first and second inflatable cuffs 50, 51. A manometry balloon can be located near the end of the inflation tubing 58, adjacent to the port 60, e.g., the valve or connector. The balloon can be derived from material having a similar compliance as the inflatable cuff material.

The bypass device 18 also includes an auxiliary tube 70 fluidly connected with the flexible tube 20. The auxiliary tube 70 extends from the flexible tube 20 at a location about the midpoint between the first axial end 24 and the second axial end 26 or at a location about the midpoint between the inflatable cuffs 50, 51. In some embodiments, the auxiliary tube 70 extends radially from about the midpoint between the first axial end 24 and the second axial end 26 of the tube 20. The auxiliary tube 70 can be configured to provide a passage that is in fluid communication with the primary tube passage 38. The auxiliary tube 70 can include an external access port 74 fluidly connected to the auxiliary tube passage and tube passage 38 so that the device 18 can be flushed and nutritional supplements and/or therapeutics can be provided into the bowel 84 if necessary. The access port 74 can include a cap or plug which can be used to close the access port 74 of the auxiliary tube 70. The access port 74 can also include a connector, such as a luer connector.

The auxiliary tube 70 can be formed from the same material as the at least one inflation tubes 50, 51. In some embodiments, the auxiliary tube 70 can have an outer diameter of about 1.0 mm to about 3.0 mm. In particular embodiments, the auxiliary tube 70 can have an outer diameter of about 2.0 mm.

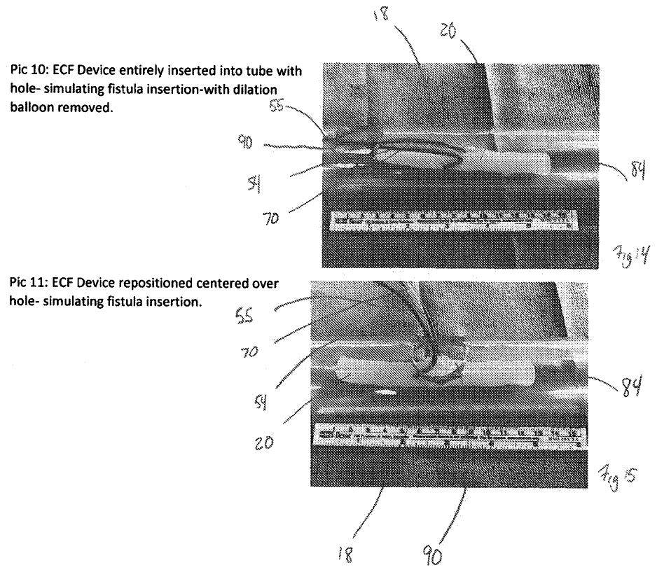

The auxiliary tube 70 and the inflation tubes 50, 51 may each be connected individually to the flexible tube 20 at a substantially 900 angle. In some embodiments, the auxiliary tube 70 and the inflation tubes 50, 51 may each be connected individually to the flexible tube 20 at angles less than 90°. In an exemplary embodiment shown in FIGS. 13-14, the inflation tubes 50, 51 and the auxiliary tube 70 are positioned substantially parallel to the centerline 22 and against an outer surface 40 of the flexible tube 20. Positioning the inflation tubes 50, 51 and the auxiliary tube 70 substantially parallel to the centerline 22 and against an outer surface 40 of the flexible tube 20 can facilitate insertion of the device 18 in a unidirectional fashion in the bowel 84 without folding of the device 18.

The bypass device 18 further includes a support wire 72 that engages the inner surface 36 of the flexible tube 20 and radially supports the flexible tube 20. The support wire 72 can extend axially along the inner surface 36 of the flexible tube 20 between the first axial end 24 and the second axial end 26. The support wire 72 can be fabricated from a medical grade surgical metal or alloy such as, but not limited to, stainless steel, titanium, titanium alloy, nichrome, or a shape memory material or alloy, such as nitinol. The support wire can have a wire diameter of about 0.2 mm to about 0.6 mm. In some embodiments, the support wire 72 can be configured to allow the flexible tube 20 to withstand external forces of about 250 mmHg without kinking or collapsing and have an internal burst pressure greater than about 250 mmHg.

FIG. 6 is an image of an example of device 18 that includes a flexible tube 20 with a support wire 72 having a coil or spiral shape. The pitch of the coil or spiral shaped support wire 72 can be varied to alter the flexibility of the tube 20. In certain embodiments, the support wire 72 is a coil having a pitch ranging from about 0.5 mm to about 10 mm. In an exemplary embodiment, a medical grade stainless steel wire coil 72 has a diameter of about 0.4 mm and a pitch of about 2 mm. The presence of the supporting wire 72 and a flexible tube 20 can allow the device 18 to be bent 360 degrees without folding or kinking.

In some embodiments, to insert the device 18 into a fistula opening 90 that would normally be too small for use given the original dimensions of the device 18, the support wire 72 can be configured in a pattern to allow the support wire 72 to alternate between an expanded position and a collapsed, or insertion position. For example, the support wire can be configured in a pattern to allow elongation, compression, and/or collapse of the support wire 72. When the support wire 72 is elongated, compressed, and/or in a collapsed position, the outer diameter of the flexible tube 20 is decreased which can allow for insertion of the device 18 into a smaller opening compared to the original dimensions, e.g., the expanded position. The elongated, compressed, and/or collapsed support wire 72 can expand to increase the outer diameter of the flexible tube 20 after insertion into the bowel 84. Optionally, the first and second inflation tubes 54, 55, the auxiliary tube 70, and/or the inflatable cuffs 50, 51 can be formed from material having a sufficient elasticity to allow the material to stretch or contract in a manner substantially similar to the positioning of the support wire 72 and return to the original expanded position if necessary.

FIGS. 10-15 illustrate an example of an ECF management bypass device 18 describe herein and its use in a method of managing fistula 90 output. An example patient 80 for receiving the device 18 is shown in FIG. 4. The patient 80 has a lower GI tract 82 including a bowel 84. An enterocutaneous fistula 90 extends through the bowel wall 89 and skin 86. As a result, intestinal enteric contents 92 such as chyme can pass from the bowel 84, through the fistula 90, and out through the skin 86.

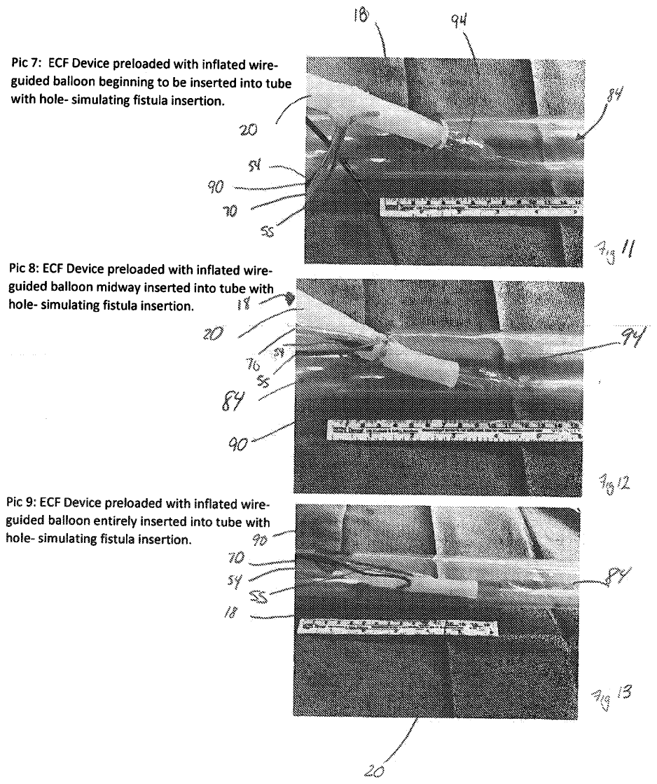

The bypass device 18 can be implanted in the bowel 84 by insertion through the fistula 90. The device 18 can be inserted into the bowel 84 manually, via a guiding catheter or via a wire-guided dilation balloon 94 following endoscopic and/or fluoroscopic interrogation of the fistula 90 tract and involved bowel 84. Prior to insertion, the tube 20 of the device 18 is preloaded with a wire-guided dilation balloon 94 (FIG. 10). The preloaded device 18 is inserted through the fistula 90 (FIG. 11) and can be inserted into the proximal or distal limb of the bowel 84 in relation to the fistula 90.

In some embodiments, the device 18 is bidirectional, wherein the first axial end 24 and the second axial end 26 are substantially identical. This allows either the first axial end 24 or the second axial end 26 of the device 18 to be inserted first into the bowel 84 via the fistula 90 opening. The bidirectional nature of the device 18 also eliminates the need to identify the direction of intestinal flow in the bowel 84 before insertion.

As shown in FIG. 13, during insertion of the device 18, the at least one inflation tube 54, 55 and the auxiliary tube 70 can be positioned parallel, or substantially parallel, to the centerline 22 of the tube 20 and against the outer surface 40 along the length of the tube 20. Positioning the at least one inflation tube 54, 55 and the auxiliary tube 70 parallel, or substantially parallel, to the centerline 22 and against an outer surface 40 of the flexible tube 20 can facilitate insertion of the device 18 in unidirectional fashion into the bowel 84, e.g., either a distal or proximal direction relative to the fistula 90. In this manner the at least one inflation tube 54, 55 and auxiliary tube 70 acquire a low profile that can reduce the need to distort, fold, or bend the device 18 for insertion and further enable insertion through smaller fistulas compared to a device having a rigid inflation and/or auxiliary tube.

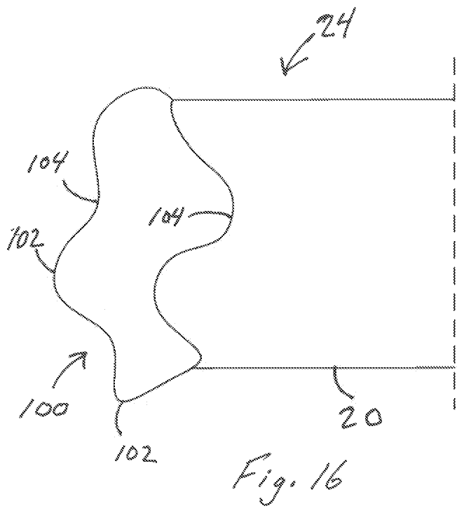

Once the first axial end 24 and the second axial end 26 of the flexible tube 20 is inserted within the bowel 84, the guide catheter or wire-guided dilation balloon 94 can be removed (FIG. 14), if necessary. Radiopaque markers can be incorporated in the tube 20 to help precisely locate the tube 20 along the bowel 84. The device 18 can then be repositioned within the bowel 84 to align the flexible tube 20 at about the midpoint in length with the fistula opening 90 (FIG. 15). For example, the inflation tubes 54, 55 and the auxiliary tube 70 can be used as central tether to pull the device into the desired position relative to the fistula opening 90. The second axial end 26 may be further guided distally using a guide wire through an eyelet in the wavy edge. In this manner, the inflation tubes 54, 55 and auxiliary 70 tube are positioned to extend substantially radially from the flexible tube 20 and out through the fistula opening 90.

A fistula opening 90 to be managed in accordance with a method described herein may be smaller than would allow for insertion of the device 18. Therefore, in some embodiments the method can include elongating, compressing and/or collapsing the device 18 prior to insertion through the fistula opening 90. Elongating, compressing and/or collapsing the device 18 can include elongating, compressing and/or collapsing the wire support 72, the at least one inflation tubes 54, 55, the auxiliary tube 70, and/or the inflatable cuffs 50, 51, as necessary. The elongated, compressed and/or collapsed device 18 can then be expanded to the original dimensions after being inserted in the bowel 84.

After the device 18 has been inserted and the inflatable cuffs 50, 51 are inflated, one or more additional step may be performed. For example, the at least one inflation tube 54, 55 and auxiliary 70 tube exiting the fistula 90 can be retained at the patient's skin 86 using an external bumper (not shown) to prevent migration of the device 18. As another example, dressings may be placed over the fistula 90 tract opening after the device 18 has be positioned. In addition, a vacuum assisted closure (VAC) device may be applied to the fistula opening 90.

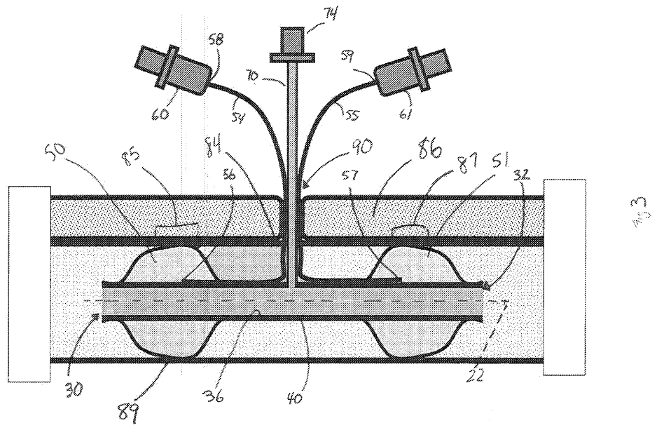

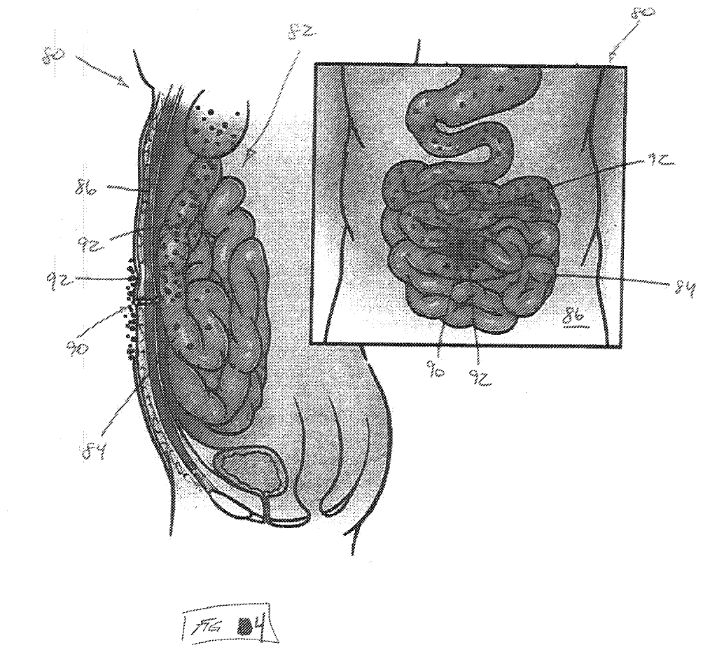

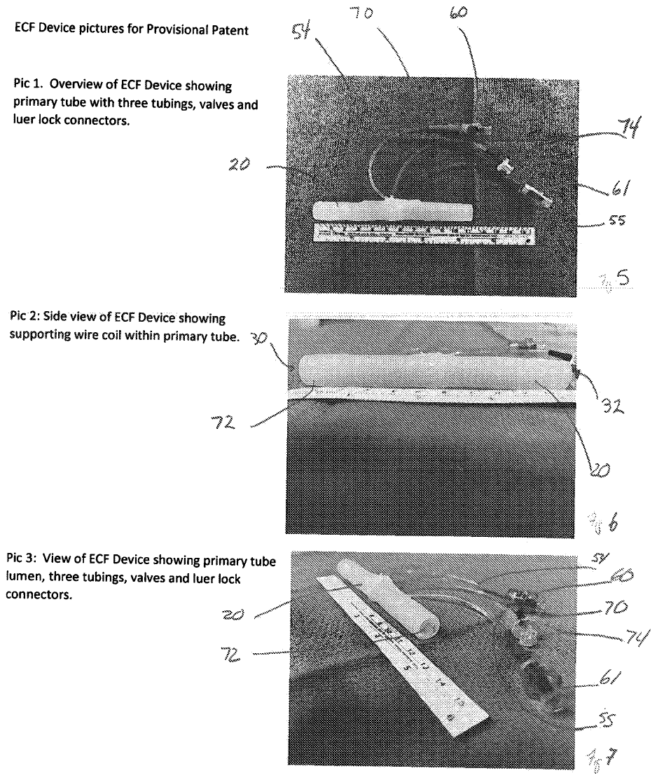

Referring again to FIG. 3, once the device 18 is inserted in the bowel 84 via a fistula opening 90 and properly repositioned as necessary, a first section of the bowel 85 located upstream of the fistula 90 opening overlaps the first inflatable cuff 50, and a second section of the bowel 87 located downstream of the fistula 90 opening overlaps the second inflatable cuff 51.

Ports 60, 61 are used to inflate the inflatable cuffs 50, 51 into engagement with the bowel wall 89 in a fluid-tight manner, which also helps to seal the first axial end 24 and the second axial end 26 of the tube 20 to the intestine 84. The port 60 used for inflation can be fluidly connected to a fluid supply via a valve and a connector, e.g., a Luer lock connector, secured to the end of the inflation tube 58.

Upon inflation of the cuffs 50, 51, the flexible tube 20 generally conforms to the path/geometry of the bowel 84. In some embodiments, the inflatable cuffs 50, 51 are inflated to an internal fluid pressure effective to create a fluid seal with the wall 89 of the bowel 84 while preventing ischemia. In some embodiments, the inflatable cuffs 50, 51 are inflated to a desired internal fluid pressure using fluoroscopic or manometry guidance.

Although not shown, any additional cuffs along the length of the tube 20 are also inflated into engagement with the intestine 84 at this time to further fix the tube 20 in position. Once inflated to a desired internal fluid pressure, the cuffs 50, 51 at the first axial end 24 and the second axial end 26 create a fluid seal between each of the inflatable cuffs 50, 51 and the bowel 84 and prevent fluid flow around the tube 20 and, thus, the contents 92 are only capable of flowing through the passage 38. Since the tube 20 extends both upstream and downstream of the fistula 90, and the cuffs 50, 51 are fluidly sealed to the bowel wall 89 at the axial ends 24, 26, the device 18 enables the contents 92 of the intestine 84 to bypass the fistula 90 and continue flowing downstream through the remainder of the GI tract 82. In accordance with the method, the bypass device 18 provides improved continuity in the bowel 84, thereby reducing fluid loss, allowing for fluid and nutrient absorption, reducing skin irritation, and improving wound management.

Following insertion and inflation, the fluid pressure of the inflatable cuffs 50,51 can be monitored to ensure the inflatable cuffs 50, 51 maintain a fluid seal with the bowel 84. While the device 18 is maintained in the bowel 84, the external port 74 of the auxiliary tube 70 can be attached to a syringe, e.g., where the external port 74 includes a syringe port, thereby allowing the tube 20 to be flushed as necessary.

Insertion of the bypass device 18 into the bowel 84 can be temporary and when necessary, the device 18 can be deflated via a port 60, 61 and removed through the fistula opening 90 once the fistula is adequately healed. In some embodiments, once inserted into the bowel 84, the device 18 is configured to function effectively for about three months. In some embodiments, each of the inflatable cuffs 50, 51 of the device 18 can be deflated via the respective port 60, 61 and exchanged for a new device 18. For example, a new device 18 can be inserted into a patient 80 in a situation where the fistula 90 has yet to heal completely and the inserted device 18 is approaching, or has past, the expected service life of the device 18. In some embodiments, to aid in removal, the device 18 can be elongated, compressed, and/or collapsed prior to removal through the fistula opening 90.

In alternative embodiments, to aid in removal, the device can be configured to allow the wire support 72 to be stripped from the device 18 in a manner resulting in a flaccid, collapsible tube.

In alternative embodiments, the auxiliary tube 70 and at least one inflation tubes 54, 55 can then be cut and the device 18 allowed naturally pass through the GI tract 82 and out of the patient 80.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

1: A device for bypassing an enterocutaneous fistula in a patient, comprising:
a flexible tube extending from a first axial end to a second axial end and defining a passage extending the entire length of the tube;
inflatable cuffs located at each of the first axial end and the second axial end of the tube, the inflatable cuffs configured to seal the first axial end and the second axial end to the internal wall of a bowel;
at least one inflation tube fluidly connected to the inflatable cuffs for supplying inflation liquid to and from the inflatable cuffs, wherein the inflatable cuffs are configured to inflate to a fluid pressure effective to create a fluid seal to the internal wall of the bowel;
an auxiliary tube fluidly connected with the flexible tube, the auxiliary tube extending from the flexible tube at a location between the first axial end and the second axial end;
a support wire engaging an inner surface of the flexible tube and extending axially along the inner surface between the first axial end and the second axial end.

2: The device of claim 1, wherein the inflatable cuffs provide a fluid barrier against the internal bowel wall while substantially minimizing ulceration or abrasion of the bowel.

3: The device of claim 1, wherein the inflatable cuffs are configured to allow an internal fluid pressure range of about 30 mmHg to about 50 mmHg at a volume of about 20 mL when implanted and confined in the bowel.

4: The device of claim 1, wherein the inflatable cuffs allow for expansion without a substantial increase in internal fluid pressure.

5: The device of claim 1, wherein the fluid pressure effective to create a fluid seal to the internal wall of the bowel allows for substantially normal intestinal blood perfusion, peristalsis, and mucosal secretion in the bowel.

6: The device of claim 1, wherein the inflatable cuffs are configured to:
allow the bowel to deform the inflatable cuffs rather than the cuff causing deformation of the bowel; and/or
distribute a substantially equal radial force to the internal wall of the bowel in contact with the inflatable cuffs; and/or
seal the first axial end to the internal wall of a bowel upstream of the fistula and the second axial end to the internal wall of the bowel downstream of the fistula.

7. (canceled)

8. (canceled)

9: The device of claim 1, wherein the inflatable cuffs are located circumferentially about the flexible tube.

10: The device of claim 1, wherein the at least one inflation tube extends from the inflatable cuff along the passage of the flexible tube and extends radially from the outer surface of the flexible tube at about the midpoint between the first axial end and the second axial end.

11: The device of claim 1, the inflatable cuffs having an axial width ranging from about 20 mm to about 60 mm.

12: The device of claim 1, wherein at least one inflation tube having a port fluidly connected to the inflation cuffs.

13. (canceled)

14: The device of claim 1, comprising a first inflation tube fluidly connected to a first inflation cuff and a second inflation tube fluidly connected to a second inflation cuff.

15: The device of claim 1, wherein at least one inflation tube comprising a manometer.

16. (canceled)

17. (canceled)

18: The device of claim 1, wherein the auxiliary tube extends from the flexible tube at about the midpoint between the first axial end and the second axial end and at a location between the inflatable cuffs.

19. (canceled)

20: The device of claim 1, wherein the at least one inflation tube and the auxiliary tube are positioned substantially parallel to a centerline and against an outer surface of the flexible tube to facilitate insertion of the device in a unidirectional fashion into the bowel without folding of the device.

21: The device of claim 1, wherein the support wire is a wire coil of medical grade stainless steel or a shape memory alloy having a pitch ranging from about 0.5 mm to about 10 mm provided within the passage of the flexible tube.

22. (canceled)

23. (canceled)

24: The device of claim 1, wherein the support wire is configured in a pattern to allow the support wire to move from an expanded position to a collapsed or insertion position, wherein the collapsed or insertion position decreases the outer diameter of the flexible tube.

25: The device of claim 24, wherein the support wire is configured in a pattern:
to allow elongation, compression, and/or collapse of the support wire to decrease the outer diameter of the flexible tube prior to insertion of the device through the fistula; and/or
to allow expansion of the support wire to increase the outer diameter of the flexible tube after insertion into the bowel.

26-29. (canceled)

30: The device of claim 1, the flexible tube having a length ranging from about 10.0 cm to about 20.0 cm, an outer diameter ranging from about 8.0 mm to about 26.0 mm, and/or an inner lumen diameter ranging from about 6.0 mm to about 20.0 mm.

31-34. (canceled)

35: The device of claim 1, wherein the shape of first axial end and/or second axial are configured to reduce the risk of bowel obstruction by having at least one of a blunt or square edge, a beveled tip or edge, or wavy edge forming two, three or more protrusions.

36: A method of managing fistula output using the fistula management device of claim 1, the method comprising the steps of:
(a) inserting the first and second axial ends of the flexible tube in a bowel via a fistula opening such that: (i) a first section of the bowel located upstream of the fistula opening overlaps a first one of the inflatable cuffs; and (ii) a second section of the bowel located downstream of the fistula opening overlaps a second one of the inflatable cuffs; and
(b) creating a fluid seal between each of the inflatable cuffs and the bowel by inflating each of the inflatable cuffs into engagement with the internal wall of the bowel.

37-50. (canceled)
